(19) United States
(12) Patent Application Publication
(10) Pub. No.: US 2006/0206077 A1
(43) Pub. Date: Sep. 14, 2006

(54) Absorbent article having barrier sheet against the transfer of the skin care composition

(75) Inventors: Raphael Warren, Amberly Village, OH (US); Ryo Minoguchi, Blue Ash, OH (US)

Correspondence Address: THE PROCTER & GAMBLE COMPANY;INTELLECTUAL PROPERTY DIVISION, WINTON HILL BUSINESS CENTER - BOX 161, 6110 CENTER HILL AVENUE, CINCINNATI, OH, 45224, US

(73) Assignee: The Procter & Gamble Company

(21) Appl. No.: 11/079,043

(22) Filed: Mar. 14, 2005

(51) Int. Cl. A61F 13/15

(52) U.S. Cl. 604/385.02; 604/359; 604/385.05

(57) ABSTRACT

An absorbent article having a fluorochemical free barrier sheet. The absorbent article has a body surface and a garment surface and an absorbent core. At least a portion of the absorbent article has a skin care composition provided thereon. The absorbent article has a fluorochemical free barrier sheet that is resistant to transfer of the skin care composition thereon in such a manner as to leave a visually-perceptible mark or stain.

### FIELD OF THE INVENTION

This application relates to absorbent articles that comprise a barrier sheet that is resistant to transfer of a skin care composition thereon.

### BACKGROUND OF THE INVENTION

All manner and variety of absorbent articles for the absorption of body fluids are well known. These absorbent articles are generally effective for absorbing body fluids, however, discomforts associated with the wearing of these absorbent articles need to be solved. For example, the wearer feels uncomfortable due to friction associated with wearing/applying the product, adherence of the menses to the wearer's skin; and adherence of the surface of the product to the wearer's skin. These discomforts may lead to the wearer having a messy/dirty feeling, over hydration of the skin, itching, and skin irritation.

Thus, it would be desirable to apply skin care compositions on absorbent articles to reduce the discomfort associated with wearing absorbent articles. Oil-based skin care compositions are known to have an effects on the skin of the wearer, such as to soften, smoothen, coat, moisturize, lubricate, or cleanse the skin, as well, reduce wetting of the sweat, feces, and/or menses against the skin and/or the topsheet of the article. However, such oil-based skin care compositions tend not to remain localized, but transfers or migrates to other portions of the absorbent article. For example, when folded for packaging, a skin care composition on the topsheet of a sanitary napkin can transfer to the backsheet or the release paper covering the panty fastening adhesive, leaving an aesthetically unpleasing stain. This transfer or migration of the oil-based skin care composition to unintended portions of the absorbent article may degrade the appearance and/or quality of the absorbent article. This transfers or migration may lead to reduced overall liquid absorbency of the absorbent article and may even degrade the adhesive, causing the absorbent article not to perform as expected.

Further, the composition can transfer on to other, unintended, portions of the article. Such transfer can result in unsightly stains that can render the article aesthetically unpleasing.

Thus, there is a need for an absorbent article that is resistant to the transfer and/or migration of a skin care composition deposited thereon.

### SUMMARY OF THE INVENTION

The present invention relates to an absorbent article having a fluorochemical free barrier sheet. The absorbent article has a body surface and a garment surface and an absorbent core. At least a portion of the absorbent article has a skin care composition provided thereon. The absorbent article has a fluorochemical free barrier sheet that is resistant to transfer of the skin care composition thereon in such a manner as to leave a visually-perceptible mark or stain.

### BRIEF DESCRIPTION OF THE DRAWINGS

While the specification concludes with claims particularly pointing out and distinctly claiming the subject matter which is regarded as forming the present invention, it is believed that the invention will be better understood from the following description which is taken in conjunction with the accompanying drawings in which:

FIG. 1 is a top plan view of one embodiment of an absorbent article in the form of a sanitary napkin;

FIG. 2 is a lateral cross-sectional view taken along line 2-2 of the sanitary napkin shown in FIG. 1;

FIG. 3 is a lateral cross-sectional view taken along line 3-3 of FIG. 1 through the center portion of one of the flaps;

FIG. 4 is a top plan view of the sanitary napkin shown in FIG. 1 showing areas where skin care composition an be applied;

FIG. 5 is a top plan view of one embodiment of a wrapper comprising a barrier sheet in an opened position with the sanitary napkin shown in FIG. 1;

FIG. 6 is a top plan view of the wrapper and the sanitary napkin shown in FIG. 5 with the flaps folded over the topsheet of the sanitary napkin;

FIG. 7 is a perspective view of the wrapper shown in FIG. 5;

FIG. 8 is a fragmented perspective view of the wrapper shown in FIG. 7 with the free end of the flap fastening cover folded back away from the main wrapper sheet;

FIG. 9 is a cross-sectional view (taken along the centerline) of the wrapper with the sanitary napkin therein in a folded configuration.

### DETAILED DESCRIPTION OF THE INVENTION

A. Absorbent Article

As used herein “absorbent article” refers to devices which are placed against the skin of a wearer to absorb and contain the various exudates discharged from the body. As used herein “disposable” is used to describe absorbent articles which for a single use and are not intended to be laundered, restored or otherwise reused as an absorbent article after a single use. Examples of disposable absorbent articles include feminine hygiene articles such as tampons, interlabial devices, sanitary napkins and panti-liners, diapers, incontinence briefs, diaper holders, training pants, and the like. As used herein “tampon” refers to any type of absorbent structure which is inserted into the vaginal canal or other body cavities for the absorption of fluid therefrom. The basic tampon structures are described in U.S. Pat. No. 1,926,900 issued to Haas on Sep. 12, 1933; U.S. Pat. No. 1,946,911 issued to Haas on Jul. 3, 1934; and U.S. Pat. No. 3,322,123 issued to Giswold, et al. on May 30, 1967. As used herein “interlabial absorbent article” refers to an absorbent device that is insertable into the interlabial space of a female wearer for catamenial purposes, incontinence barrier, or both. Suitable interlabial absorbent articles are disclosed in, e.g., U.S. Pat. No. 5,762,644 entitled “Toilet-Disposable Absorbent Interlabial Device” issued to Osborn, et al. on Jun. 9, 1998; PCT Publication No. WO 98/29078 entitled “Thin Comfortable Interlabial Absorbent Structure” published in the name of Osborn, et al. on Jul. 9, 1998; U.S. Pat. Des. No. 404,814 entitled “Interlabial Absorbent Device” issued to Mayer on Jan. 26, 1999; U.S. Pat. No. 6,270,486 entitled “Absorbent Interlabial Device” issued to Brown, et al. on Aug. 7, 2001. As used herein, the terms “panty liner” or “panti-liner” refer to absorbent articles that are less bulky than sanitary napkins which are generally worn by women between their menstrual periods. Suitable absorbent articles are disclosed in, e.g., U.S. Pat. No. 4,738,676 entitled “Pantiliner” issued to Osborn on Apr. 19, 1988. As used herein “diaper” refers to an absorbent generally worn by infants, and incontinent persons that is worn about the lower torso of the wearer. Suitable diapers are disclosed in, e.g., U.S. Pat. No. 3,860,003 issued to Buell on Jan. 14, 1975; U.S. Pat. No. 5,151,092 issued to Buell et al. on Sep. 29, 1992; U.S. Pat. No. 5,221,274 issued to Buell et al. on Jun. 22, 1993; and U.S. Pat. No. 5,554,145 issued to Roe et al. on Sep. 10, 1996. As used herein “incontinence article” refers to pads, undergarments, inserts for absorbent articles, capacity boosters for absorbent articles, briefs, bed pads, and the like regardless of whether they are worn by adults or other incontinent persons. Suitable incontinence articles are disclosed in, e.g., U.S. Pat. No. 4,253,461 issued to Strickland, et al. on Mar. 3, 1981; U.S. Pat. Nos. 4,597,760 and 4,597,761 issued to Buell; the above-mentioned U.S. Pat. No. 4,704,115; U.S. Pat. No. 4,909,802 issued to Ahr, et al.; U.S. Pat. No. 4,964,860 issued to Gipson, et al. on Oct. 23, 1990; and PCT Publication No. WO 92/11830 published by Noel, et al. on Jul. 23, 1992. As used herein “training pants” refers to disposable garments having fixed sides and leg openings. Training pants are placed in position on the wearer by inserting the wearer's legs into the leg openings and sliding the training pant into position about the wearer's lower torso. Suitable training pants are disclosed in, e.g., U.S. Pat. No. 5,246,433, issued to Hasse, et al. on Sep. 21, 1993.

“Body surface,” as used herein, means that surface of the article or component which is intended to be worn toward or adjacent to the body of the wearer. “Garment surface,” as used herein, means the surface on the opposite side of the body surface. As used herein, the term “body contacting surface” of an absorbent article is one or more surfaces of any article components that contact the wearer at some time during the wear period. Body contacting surfaces include, but are not limited to, portions of the topsheet, flaps, leg cuffs, waist region, side panels, fastening tabs, etc., which contact a wearer during use.

The term “joined”, as used herein, encompasses configurations in which an element is directly secured to another element by affixing the element directly to the other element; configurations in which the element is indirectly secured to the other element by affixing the element to intermediate member(s) which in turn are affixed to the other element; and configurations in which one element is integral with another element, i.e., one element is essentially part of the other element.

The term “longitudinal”, as used herein, refers to a line, axis or direction in the plane of the sanitary napkin that is generally aligned with (e.g., approximately parallel to) a vertical plane which bisects a standing wearer into left and right body halves when the sanitary napkin is worn. The terms “transverse” or “lateral” used herein, are interchangeable, and refer to a line, axis or direction which lies within the plane of the sanitary napkin that is generally perpendicular to the longitudinal direction.

As used herein, the terms “migrate”, “migration”, or “migrating” mean the skin care composition moves from one place to another place by way of movement on a material or permeation through an intervening material.

As used herein, the term “transfer” when used in the context of the skin care composition, refers to the skin care composition moving from one area of the absorbent article to another area on the absorbent article not by way of migration but by way of direct contact with the skin care composition, such as in a blotting effect.

As used herein, the term “substantially prevent” means that a barrier sheet prevents the migration or transfer of substantial amounts of a skin care composition into or onto the barrier sheet. The prevention level (i.e., durability effect) of the barrier sheet against the skin care composition can be assessed by an Accelerated Migration Test. The Accelerated Migration Test is practiced by the following steps; (1) lay 120×120 mm of a barrier sheet material on an aluminum foil sheet, (2) place a catamenial pad having a skin care composition on the topsheet against the barrier sheet with the topsheet facing and in direct contact with the barrier sheet; (3) place a weight on the catamenial pad, the weight having a weight of at least 900 g, preferably 1200 g, and more preferably 2000 g; (4) hold the Migration Test Sample in an incubator at 40±1° C. for 24 hours ±½ hour; (5) after the designated time, remove the weight and test catamenial pad by lifting without any motion of shear; (6) observe the barrier sheet for any visually perceptible transfer of skin care composition, such as in the form of a visible oil stain on the barrier sheet. If a visible oil stain on the barrier test sheet is observed at the time of observation, it is assessed that the migration or transfer of a skin care composition into or onto the barrier sheet has occurred and the barrier sheet has a durability defect. Optionally, if a stain is not clearly visually perceptible after step (6), another step can be made: (7) Transfer the barrier sheet onto a clean dark surface. If a visible oil stain on the barrier test sheet is observed at the time of observation, it is assessed that the migration or transfer of a skin care composition into or onto the barrier sheet has occurred and the barrier sheet has a durability defect.

As used herein, the term “visually perceptible transfer” is meant an amount of transfer of skin care composition sufficient that a human viewer can visually discern the transferred skin care composition with the unaided eye (excepting standard corrective lenses adapted to compensate for near-sightedness, farsightedness, or stigmatism, or other corrected vision) in lighting at least equal to the illumination of a standard 100 watt incandescent white light bulb at a distance of 1 meter.

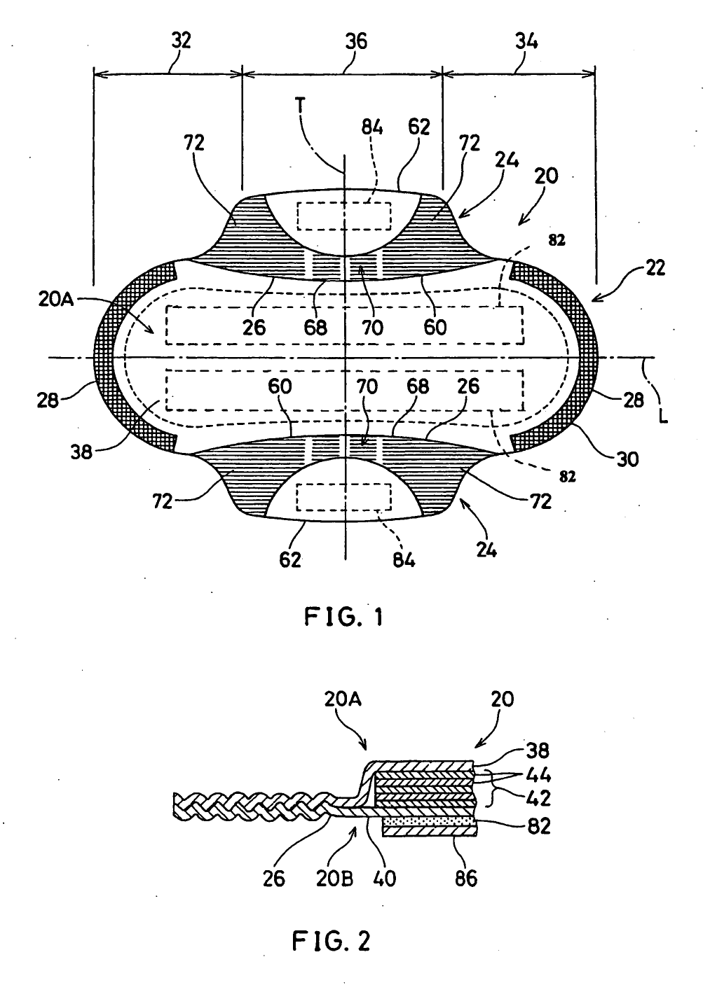

FIG. 1 depicts a sanitary napkin which is one type of disposable absorbent article used for external wear about the pudenda region of the wearer. As shown in FIG. 1, the sanitary napkin 20 comprises main body portion 22, and two optional flaps 24. The sanitary napkin 20 has two surfaces, a body surface 20A and a garment surface 20B. FIG. 1 is viewed from its body surface 20A. The sanitary napkin 20 has a principal longitudinal centerline L and a principal transverse centerline T. The main body portion 22 has two spaced apart longitudinal edges 26, two spaced apart transverse or end edges 28, which together form the periphery 30 of the main body portion. The main body portion 22 also has a first end region 32 and second end region 34 with a central region 36 is disposed between. The main body portion 22 of the sanitary napkin 20 can be of any thickness, including relatively thick, intermediate thickness, relatively thin, or even very thin (or “ultra thin” as described in U.S. Pat. Nos. 4,950,264 and 5,009,653 issued to Osborn.).

FIG. 2 shows the individual components of the main body portion 22 of the sanitary napkin 20, including a liquid pervious topsheet 38, a liquid impervious backsheet 40, and an absorbent core 42 which can have individual layers 44 as a composite positioned between the topsheet 38 and the backsheet 40. Panty fastening adhesive 82 can be covered by release strip 86.

A suitable topsheet 38 may be manufactured from a wide range of materials such as woven and nonwoven materials, apertured formed thermoplastic films: apertured plastic films; hydroformed thermoplastic films; porous foams; reticulated foams; reticulated thermoplastic films; and thermoplastic scrims. Suitable woven and nonwoven materials can be comprised of natural fibers, synthetic fibers, or from a combination of natural and synthetic fibers. The nonwoven web may be manufactured by a wide number of known techniques including spunbonded, carded, wet-laid, melt-blown, hydroentangled, combinations of the above, or the like. A suitable the topsheet 38 comprises an apertured formed film made in accordance with U.S. Pat. No. 4,342,314 issued to Radel, et al. and U.S. Pat. No. 4,463,045 issued to Ahr, et al., which is marketed on sanitary napkins as the DRI-WEAVE topsheet by The Procter & Gamble Company of Cincinnati, Ohio. Such an apertured film can be obtained as product No. X-5652 from Tredegar Film Products of Terre Haute, Ind. The body surface of the topsheet 38 is hydrophilic or can be made hydrophilic by treating it with a surfactant, so that liquids will be transferred through the topsheet more readily. Such surfactant are described in U.S. Pat. No. 4,950,254 issued to Osborn; U.S. Pat. No. 4,988,344 entitled “Absorbent Articles with Multiple Layer Absorbent Layers” issued to Reising, et al on Jan. 29, 1991; and U.S. Pat. No. 4,988,345 entitled “Absorbent Articles with Rapid Acquiring Absorbent Cores” issued to Reising on Jan. 29, 1991.

The backsheet 40 is impervious to liquids and can comprise a thin plastic film, a woven or nonwoven material, polymeric films such as thermoplastic films of polyethylene or polypropylene, or composite materials such as a film-coated nonwoven material. A suitable backsheet 40 is a polyethylene film having a thickness of from about 0.012 mm (0.5 mil) to about 0.051 mm (2.0 mils). Exemplary polyethylene films are manufactured by Clopay Corporation of Cincinnati, Ohio, under the designation P18-1401 and by Tredegar Film Products of Terre Haute, Ind., under the designation XP-39385. A suitable breathable backsheet material is a laminate of an apertured film such as that described in U.S. Pat. No. 3,929,135 issued to Thompson which is inverted so that the smaller openings of the tapered capillaries face the absorbent core 42 which is adhesively laminated to a microporous film such as that described in Exxon's U.S. Pat. No. 4,777,073.

The absorbent core 42 may be manufactured in a wide variety of sizes and shapes (e.g., rectangular, oval, hourglass, “T” shaped, dog bone, asymmetric, etc.). The absorbent core may include any of a wide variety of liquid-absorbent materials, such as comminuted wood pulp (airfelt), creped cellulose wadding; meltblown polymers (coform; chemically stiffened, modified or cross-linked cellulosic fibers); synthetic fibers (crimped polyester fibers); peat moss; tissue wraps; tissue laminates; absorbent foams; absorbent sponges; superabsorbent polymers; absorbent gelling materials; and the like, and mixtures of these. The configuration and construction of the absorbent core 42 may also be varied (e.g. varying caliper zones; profiled to be thicker in the center; hydrophilic gradients; gradients of the absorbent composite, superabsorbent gradients; lower average density zone, lower average basis weight zones, acquisition zones; or layers). The size and total absorbent capacity of the absorbent core may be varied to accommodate different uses such as diapers, incontinence pads, pantiliners, regular sanitary napkins, and overnight sanitary napkins, and to accommodate wearers ranging from infants to adults.

The absorbent core 42 can comprise a multi-bonded air laid nonwoven material comprising about 52% cellulose fibers, about 20% bi-component fibers, about 25% superabsorbent hydrogel-forming material particles, and about 3% latex binder. The absorbent core 42 can have a basis weight of about 125 g/yd² (about 150 g/m²), including the particles of absorbent gelling material. Such a multibonded airlaid nonwoven material is obtained in roll form as product 915000X313 from Merfin Hygienic Products.

The absorbent core can include other absorbent components, such as, dusting layer, a wicking or acquisition layer, or a secondary topsheet for increasing the wearer's comfort. An acquisition component 44 may either be a separate component positioned between the topsheet 38 and the absorbent core 42, or it may comprise part of the topsheet 38 or part of the absorbent core 42. The acquisition component 44 can be made from any materials suitable, including wovens and nonwovens made from synthetic, natural or a combination of natural and synthetic fibers. The acquisition component 44, if nonwoven, can be made by a number of different processes including but not limited to air laid, wet laid, meltblown, spunbonded, carded, thermally bonded, air-through bonded, powder bonded, latex bonded, solvent bonded, spunlaced, and combinations of the foregoing.

An acquisition component 44 can be comprises of a laminate of two nonwoven materials. The uppermost layer can comprise a 19 g/yd² (22.5 g/m²) spunbonded polypropylene nonwoven material referred to as product No. 065MLPV60U (or “P-9”) obtained from Fiberweb, North America of Washougal, Wash. The underlying layer can comprise a multi-bonded air laid nonwoven material that is thermally bonded using powder bonding and latex bonding. This multi-bonded air laid nonwoven material can comprise about 77% cellulose fibers, about 20% powder binder, and about 3% latex binder (1.5% sprayed on each side of the web) and has a basis weight of about 50 g/yd² (about 60 g/m²). Such a multi-bonded air laid nonwoven can be obtained as product No. 90830X312 from Merfin Hygienic Products, Ltd. of Delta, British Columbia, Canada. A low density latex bonded air laid material suitable for this purpose is a material having a basis weight of about 80 g/m² known as product No. FG413MHB, which is obtained from Walkisoft, USA of Mt. Holly, N.C.

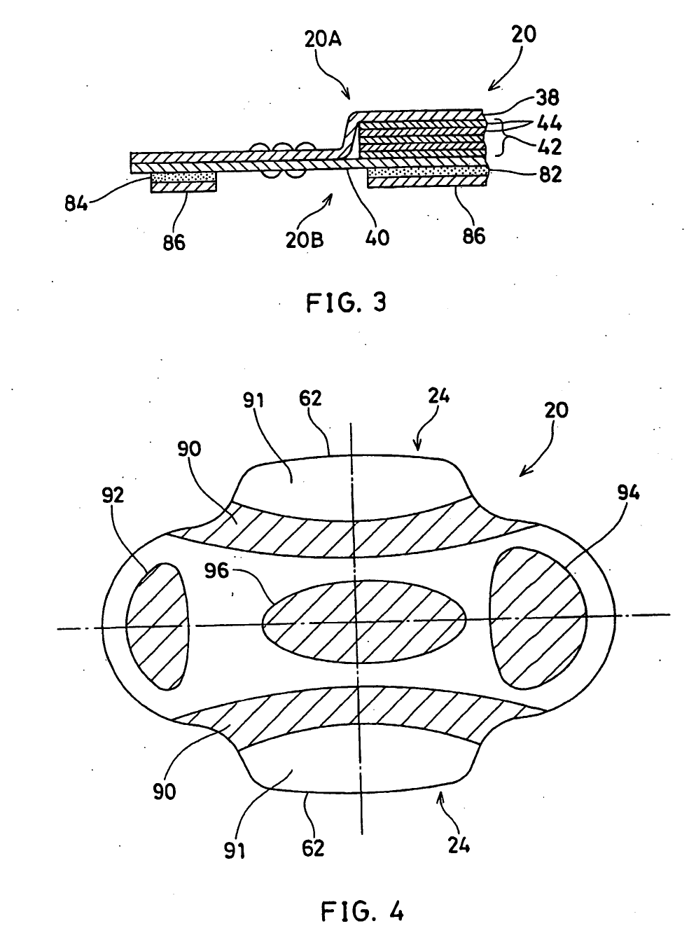

The topsheet 38, the backsheet 40, and the absorbent core 42 may be layered or “sandwich” configurations as in FIG. 1-3 and wrapped or “tube” configurations. In the embodiment shown in FIGS. 2 and 3 having the acquisition component 44, the garment-facing side of the topsheet 38 can be joined to the body-facing side of the absorbent core. If the absorbent core 42 is layered structure, each layer 44 can be joined. The acquisition component 44 can be joined to the absorbent core 42. The backsheet 40 is joined to the garment-facing side of the absorbent core 42. The components of the main body portion 22 of the sanitary napkin 20 can be joined to be any means know in the art including but not limited to adhesive means, heat bonds, pressure bonds, ultrasonic bonds, dynamic mechanical bonds, or any other suitable attachment means or combinations known in the art. A uniform continuous layer of adhesive, a patterned layer of adhesive, or an array of separate lines, spirals, or spots of adhesive may be used. Adhesives which have been found to be satisfactory are manufactured by H. B. Fuller Company of St. Paul, Minn. under the designation HL-1258 or H-2031

The sanitary napkin 20 shown in FIGS. 1-3, as discussed above, comprises an optional pair of flaps 24 that are joined to the main body portion 22 in any suitable manner. The flaps 24 extend laterally outward beyond the longitudinal side edges 26 of the main body portion 22 from their proximal edges 60 to their distal edges 62. As shown in FIGS. 1-3, the flaps 24 are integral with the main body portion 22 comprised of extensions of the topsheet 38 and backsheet 40. However, flaps 24 can comprise separate components that are joined to the main body portion 22 along a juncture, such as lines of juncture 68 depicted by concave inwardly-oriented regions or lines.

The flaps 24 can be in any suitable configuration, such as, described in Reexamined Pat. No. B1 4,589,876 entitled “Sanitary Napkin”, issued to Van Tilburg, Certificate of Reexamination issued Apr. 27, 1993; U.S. Pat. No. 4,687,478 entitled “Shaped Sanitary Napkin With Flaps”, which issued to Van Tilburg on Aug. 18, 1987; U.S. Pat. No. 5,389,094 entitled “Absorbent Article Having Flaps and Zones of Differential Extensibility” issued to Lavash, et al. on Feb. 14, 1995; U.S. Pat. No. 5,558,663 entitled “Absorbent Article Having Undergarment Covering Components With Zones of Extensibility” issued to Weinberger, et al. on Sep. 24, 1996.”

When the sanitary napkin 20 is worn by the wearer, the flaps 24 are folded under the wearer's undergarment. The flaps 24 are typically folded along or adjacent the proximal edges 60 or may have a deformed region that forms a hinge 70 between the main body portion 22 and at least a portion of the flaps 24. The sanitary napkin 20 can have at least one zone of extensibility 72 for relieving the stresses on the flaps 24 when they are folded around a panty crotch. These are described in PCT publication WO 97/12576 published on Apr. 10, 1997 titled “Absorbent Article Having Flaps With A Deformed Hinge And Zones Of Extensibility.”

The garment surface 20B of the sanitary napkin 20 may include central pad fastener 82, commonly referred to as panty fastening adhesive, for securing the sanitary napkin in the crotch region of the wearer's undergarment. The flaps 24 can be maintained in position by attaching the flaps 24 to the undergarment, or to the opposing flap by a flap fastener 84, which is provided on the garment surface 20B of the flap 24. Any types of fasteners known in the art can be used, such as, adhesive fasteners and mechanical fasteners or a combination thereof. The adhesive fasteners can be hot melt pressure-sensitive adhesives selected from the group consisting of styrene rubber based, styrene butadiene based, and styrene isoprene based. The adhesive fasteners may be covered by removable release liner 86 to keep the adhesives from sticking to extraneous surfaces prior to use. As well, the sanitary napkin 20 could be secured to the wearer's undergarment by mechanical fasteners, such as VELCRO, or U.S. Pat. No. 5,392,498 entitled “Non-Abrasive Skin Friendly Mechanical Fastening System” issued to Goulait, et al. on Feb. 28, 1995

Skin care compositions can be applied to at least a portion of the body contacting surface of the sanitary napkin 20. The skin care composition may be applied to any portion of the body contacting surface of the sanitary napkin 20 and/or may be applied to any portion of the garment surface of the sanitary napkin 20. In some embodiments the skin care composition is not applied adjacent to the adhesive fasteners, such as the central pad adhesive fastener 82 and the flap adhesive fastener 84 because the adhesive of the fastener can be degraded by the skin care composition. The skin care composition may be applied to the body contacting surface of other types of absorbent articles including but not limited to, tampons, interlabial absorbent articles, panti-liners, incontinence articles, diapers including infant diapers, training pants, adult incontinence diapers, etc.

In the embodiment shown in FIG. 1, the skin care composition can be applied to either the entirety or portions of the topsheet 38 and, if desired to the entire area or portions of the flaps 24. The skin care composition can be applied to longitudinal end areas 92 and 94 of the sanitary napkin 20. The skin care composition can be applied to the front end area 92, which corresponds to the portion of the wearer's body around pubic hairs, the back end area 94 which corresponds to the portion of the wearer's body around anus, and the central area 96, which corresponds to the portion of the wearer's body around vulva. The skin care composition can be applied in other discontinuous configurations, such as in stripes, bands, or beads of lotion.

The absorbent article comprises a barrier sheet. A barrier sheet is a sheet or material that is resistant to the migration of the skin care composition through it, or transfer of the skin care composition onto it. The barrier sheet is preferably resistant to a visually perceptible transfer of the skin care composition, such that, a visually perceptible stain or spot from an oil-based skin care composition cannot appear on the barrier sheet if the barrier sheet is contacted by the skin care composition, especially if contacted for an extended period of time, such as when packaged prior to use. The barrier sheet can be resistant to both tactilely- and/or visually-perceptible transfer of the skin care composition thereon. As well, the barrier sheet substantially can prevent the migration of the skin care composition through the barrier sheet. The barrier sheet can be used to cover a portion of the sanitary napkin 20, such as the flap adhesive 84, the central pad adhesive 82, a portion of the topsheet 38, or a portion of the backsheet 40. In one embodiment, the barrier sheet is the release paper covering the central pad adhesive or flap adhesive. It also may be used to cover a portion of the absorbent core 42. The barrier sheet may be used for a main wrapper sheet to cover the entirety of the sanitary napkin 20.

While fluorochemical-treated papers can be used as barrier sheets, in one embodiment, a barrier sheet of the present invention is a non-fluorochemical-treated paper. That is, a barrier sheet of the present invention can be described as being fluorochemical free, or fluoropolymer free, such that the barrier sheet comprises no fluorochemicals. Such an embodiment is preferred for use in articles such as feminine hygiene articles because of the negative health effects possible with the use of fluorochemicals. For example, fluorochemical treatments on feminine hygiene articles can present potential safety issues if these materials migrate onto the article and then to the body. Such materials can also be an environmental hazard.

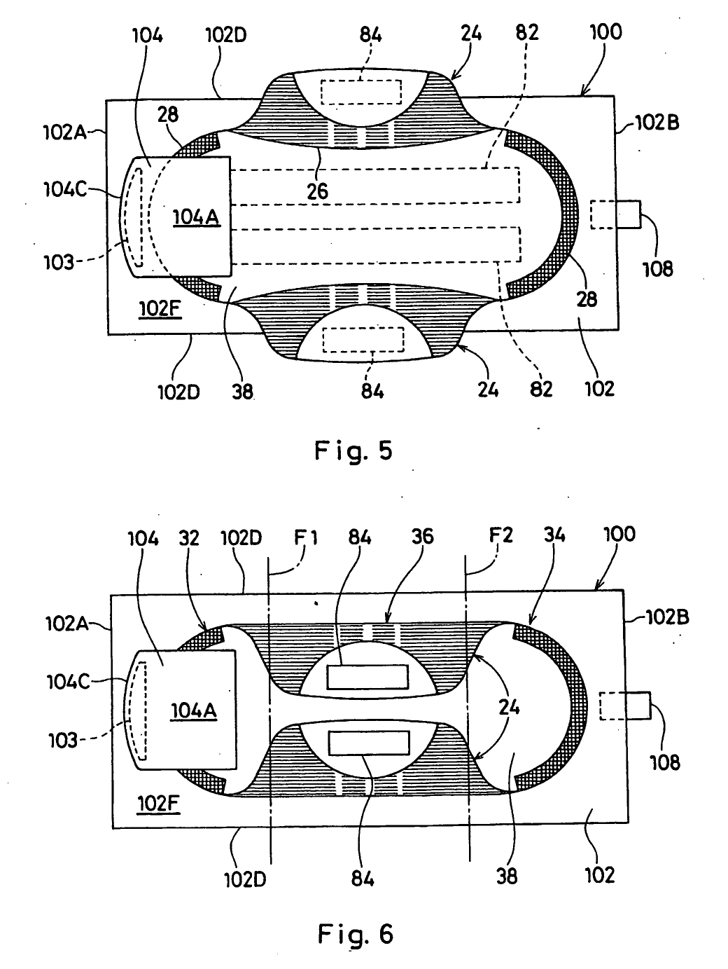

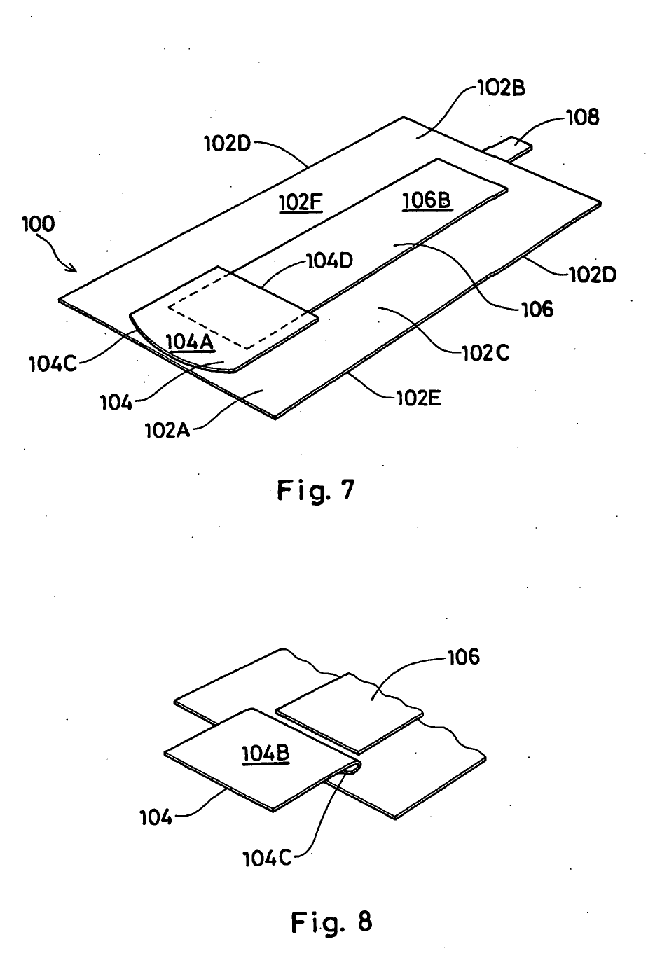

FIGS. 5-10 show embodiments of the application of the barrier sheet to the sanitary napkin 20. In the embodiments, the sanitary napkin 20 is wrapped by a wrapper 100 comprising several elements including the main wrapper sheet 102, the central pad adhesive cover 106, and the flap adhesive cover 104. These elements can comprise integral portions of a single member, or they can comprise separate components joined to a member. Each element of the wrapper 100 may comprise a barrier sheet or only a selected element may comprise a barrier sheet.

The main wrapper sheet 102 can cover the entirety of the sanitary napkin 20 and can be a barrier sheet. The main wrapper sheet 102 (or “wrapper sheet”) is the portion of the wrapper 100 which will be folded around the sanitary napkin 20 to provide an individual package for the sanitary napkin 20. As shown in FIGS. 5-7, the main wrapper sheet 102 has two surfaces; an inner surface 102F and an outer surface 102E, longitudinal edge portions 102D, a first end portion 102A and a second end portion 102B.

The central pad adhesive cover 106 may comprise a barrier sheet that protects the central pad adhesive 82 from transfer and migration of the skin care composition applied on the sanitary napkin 20. The central pad adhesive cover also resists transfer of the skin care composition, such that the cover does not have visually-perceptible deposits of the skin care composition. The central pad adhesive cover 106 is a size to cover the central pad adhesive 82. The central pad adhesive cover has two surfaces; an inner surface 106B and an opposing surface 106A. The opposing surface 106A of the central pad adhesive cover 106 can be joined to the inner surface of the main wrapper sheet 102. Likewise, the flap adhesive cover 104 may comprise a barrier sheet which is any size to cover the flap adhesive 84. In the embodiment shown in FIGS. 5 and 6, the flap adhesive cover 104 has two faces; one of which is a non-stick face (or releasable face) 104A, which is capable of releasable attachment with the flap adhesive 84, and an opposite face or side 104B.

In the embodiment shown in FIGS. 5-7, the flap adhesive cover 104 also has a pair of longitudinal edge portions, a first end portion (fixed end) 104C, and a second end portion (distal end) 104D. The first end portion 104C of the flap adhesive cover 104 is joined adjacent the first end portion 102A of the main wrapper sheet 102 by an adhesive 103. The second end portion 104D of the flap adhesive cover 104 extends toward the central portion 102C of main wrapper sheet 102 and toward the central portion 36 of sanitary napkin 20. Alternatively, the flap adhesive cover 104 may not be joined to a portion of the main wrapper sheet 102.

If the barrier sheet is used for the flap adhesive cover 104 and/or the central pad adhesive cover 106 at least one surface of the barrier sheet can be treated to be releasable. The non-stick face of the flap adhesive cover 104 A and/or inner surface the central pad adhesive cover 106 B can be treated to become releasable, such that the covers 104A or 106B will release from the adhesive when the wearer moves the sanitary napkin 20 from the wrapper 100. For the release treatment, the non stick surface of the flap adhesive cover 104 and/or inner surface of the central pad adhesive cover 106B may be provided by attaching a separate release paper or element to the surface which is treated with a non-stick material, or by treating all or a portion of the cover with a non-stick coating. The release coating may comprise any material known in the art for this purpose, with silicone coatings being preferred. If a coating is used, the coating 106 may be provided by coating the entire inner surface 102F of the main wrapper 102 or to only the zone of the main wrapper sheet 102 which will substantially contact the central pad adhesive 82. Coating the entire inside of a wrapper is disclosed in U.S. Pat. No. 5,181,610 entitled “Flexible Container with Nonstick Interior” which issued to Quick et al. on Jan. 26, 1993.

The wrapper 100 comprises an optional package fastener 108 for retaining the sanitary napkin in its folded configuration in any suitable location on the wrapper 100. The package fastener 108 is both attached releaseably to the package and is resealable, such as spots or patches of adhesive, tapes, and mechanical fasteners.

For the initial packaging of the sanitary napkin 20 in the wrapper 100, the garment surface 20B of the main body portion 22 is placed on top of the main wrapper sheet 102. The sanitary napkin 20 is positioned so that the central pad adhesive 82 lies over the central pad adhesive cover 106 on the main wrapper sheet 102. The flaps 24 can be folded such that they expose the patches of adhesive 84 disposed on the garment surface of flaps 24 and cover at least a portion of the topsheet 38. After folding the flaps 24 over the topsheet 38, the sanitary napkin 20 and the main wrapper sheet 102 will then be folded into three sections that are defined by fold axes F1 and F2 shown in FIG. 6.

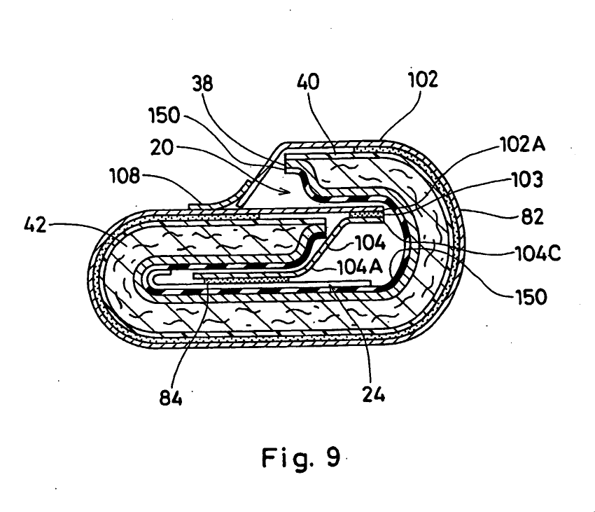

FIG. 9 depicts the package for the sanitary napkin formed by folding the wrapper 100 and sanitary napkin 20 in configuration for shipment, sale, and convenient carrying by the wearer. In FIG. 9, the skin care composition layer 150 is applied on all over the surface of the topsheet 38. The main wrapper sheet 102 also prevents the transfer and migration of the skin care composition out through the main wrapper sheet 102. As shown in FIG. 9, the first end portion 102A of the main wrapper sheet 102, along with the first end region 32 of the sanitary napkin 20 and the flap adhesive cover 104 are folded about first fold axis F1 onto the central region 36 of sanitary napkin 20. When the sanitary napkin 20 and wrapper 100 are folded in this manner, the nonstick face 104A of flap adhesive cover 104 is placed over the flap fasteners 84 and is releasably attached to each adhesive patch 84. In the folded configuration of the sanitary napkin 20 shown in FIG. 9, the flap adhesive cover 104 faces the skin care composition layer 150 and substantially protects the flap adhesive 84 and the adhesive 103 from the skin care composition. In addition, the flap adhesive cover 104 provides a connection between each flap 24 that spans the flaps 24, thereby keeping the flaps 24 in the desired position until flap adhesive cover 104 is removed. The second end portion 102B of the main wrapper sheet 102, along with the second end region 34 of sanitary napkin 20, and the tape tab 108 are then folded about the second fold axis F2. This places these components on top of the first end portion 102A of the main wrapper sheet 102 and the first end region 32 of sanitary napkin 20. By pressing the tape tab 108 onto the exterior of wrapper 100 in the position depicted in FIG. 9, the sanitary napkin 20, its flaps 24 and wrapper 100 remain in the configuration shown.

B. Skin Care Composition.

The skin care compositions of the present invention can provide for a protective, non-occlusive function, avoidance of skin exposure to materials contained in body exudates; an abrasion minimizing function to reduce skin irritation in the areas where the body contacting surface of absorbent articles contact the wearer's skin; reduce odor; or contain agents that deliver, either directly or indirectly, skin care benefits. Indirect benefits include improved removal of skin irritants such as menses, feces or urine. The composition may be in a variety of forms, including, but not limited to, emulsions, lotions, creams, ointments, salves, powders, suspensions, encapsulations, gels, and the like. Typically, the skin care composition is oil-based, herein meaning substantially free of water.

The skin care compositions may comprise: (1) one or more emollient(s) (2) one or more immobilizing agent(s) to stabilize the emollient(s) (3) one or more skin care active ingredient(s), and (4) other optional components. Although the kind, grade and content of each component of the skin care compositions are arbitrary, the skin care composition including at least one skin care active ingredient are prepared such that the compositions can be applied to an absorbent article to result in safe and effective amounts of the compositions being transferred onto the skin of a wearer of the absorbent article. Therefore, the lotion compositions preferably have a product consistency such that they are relatively immobile and localized on the wearer-contacting surface of the absorbent article at ambient conditions, are readily transferable to the wearer at body temperature, and yet are not completely liquid under extreme storage conditions. In other words, the lotion compositions are solids or semisolids at ambient conditions (about 25° C.) and/or body temperature (about 37° C.) so that the compositions are easily transferred onto the skin by way of normal contact, wearer motion, and/or body heat. The consistency of the lotion compositions can be measured according to ASTM D5 test method which involves the use of a penetrometer to measure consistency. Typically, the lotion compositions of the present invention have a consistency of from about 10 to about 300, preferably from about 20 to about 250, more preferably from about 30 to about 200, as measured at 40° C. according to the test procedure outlined in ASTM D5 test method.

The solid or semisolid consistency of the lotion compositions provide for relatively low levels of the compositions to be applied to the absorbent articles to impart the desired lotion benefits. By “semisolid” is meant that the compositions have a rheology typical of pseudoplastic or plastic liquids such that the compositions remain relatively stationary in a desired location on the absorbent article, and do not have a tendency to flow or migrate to undesired locations of the article. The solid lotion compositions of the present invention likewise can remain in a particular location and not flow or migrate to undesired locations of the article. These solid and semisolid lotion compositions have viscosities high enough to keep the compositions localized on an intended location of the article, but not so high as to impede transfer to the wearer's skin. Typically, final products of solid and semisolid lotion compositions have viscosities ranging from about 1.0×10⁶ centipoise to about 1.0×10¹⁰ centipoise under shear stress conditions of about 3×10³ dynes/cm² at 40° C. (the shear stress applied to the compositions while the absorbent article is in storage or transported at temperature conditions of about 40° C.).

However, the solid and semisolid lotion compositions can be made flowable for transfer or migration of the compositions onto the skin by applying shear stress that results in deformation of the compositions. The shear stress applied at least once during wear of the absorbent article under temperature conditions of about 40° C. is typically at about 1.0×10⁶ dynes/cm², and this shear stress can result in the lotion compositions having a viscosity of from about 1.0×10¹ centipoise to about 1.0×10⁵ centipoise. It is believed that the lotion compositions achieve the lower viscosity values under applied shear stress due to the fact that, while the compositions contain solid components, they also contain liquid materials. During wear of an absorbent article described herein, it is desirable to achieve a low viscosity for obtaining sufficient lubrication between the wearer's skin and the body contacting surface of the article to result in effective transfer of the lotion composition onto the wearer's skin. Viscosity at various shear stress can be measured using rheometers known in the art such as the Rheometer SR-2000 available from Rheometrics Incorporation.

Examples of suitable skin care compositions are disclosed in U.S. Pat. No. 5,643,588, “Diaper Having a Lotioned Topsheet”, issued to Roe, Bakes & Warner on Jul. 1, 1997; and U.S. Pat. No. 5,635,191, “Diaper Having a Lotioned Topsheet Containing a Polysiloxane Emollient”, issued to Roe & Mackey on Jun. 3, 1997; U.S. Pat. No. 5,609,587, “Diaper Having a Lotioned Topsheet Comprising a Liquid Polyol Polyester Emollient and an Immobilizing Agent”, issued to Roe on Mar. 11, 1997; U.S. Pat. No. 5,607,760, “Disposable Absorbent Article Having a Lotioned Topsheet Containing an Emollient and a Polyol Polyester Immobilizing Agent”, issued to Roe, on Mar. 4, 1997; U.S. Pat. No. 6,803,496 entitled “A Method For Maintaining or Improving Skin Health”, Elder, et al., issued on Oct. 12, 2004; U.S. Pat. No. 6,710,223 entitled “A Method For Improving Skin Condition”, Van Rijswijck, et al. issued on Mar. 24, 2004; and U.S. patent application Ser. No. 08/908,852 “Diaper Having A Lotioned Topsheet”, Roe, et al. filed on Aug. 8, 1997; U.S. patent application Ser. No. 10/152,924 “Skin Care compositions Comprising Low Concentrations of Skin Treatment Agents” Warren et al., filed May 21, 2002; U.S. patent application Ser. No. 10/444,241, “Sanitary Napkins With Hydrophobic Lotions”, Hammons et al., filed on May 23, 2003; U.S. patent application Ser. No. 10/789,967, “Sanitary Napkins with Hydrophobic Lotions” Gatto et al., filed Feb. 27, 2004; or U.S. patent application Ser. No. 10/992,383, “Sanitary Napkins with Hydrophobic Lotions” Gatto et al., filed Feb. 27, 2004.

Skin Active Ingredients:

The skin care compositions of the present invention comprise relatively low concentrations of a select combination of skin treatment agents that are capable of reducing and eliminating the occurrence of skin disorders that can result from contact between the skin and moisture-laden air, skin disorders resulting from prolonged moist human tissue that can occur from the skin being exposed to moisture or other body exudates, and/or skin disorders that are generated from contact between the skin and microbial or bacterial agents. The phrase “select combination of skin treatment agents” refers to the following combinations: a. hexamidine, zinc oxide, and niacinamide; b. hexamadine and zinc oxide; and c. hexamadine and niacinamide. The total concentration of the skin active ingredients ranges from about 0.002% to about 20%, preferably from about 0.01% to about 15%, more preferably from about 0.05% to about 15% by weight of the lotion composition.

The skin care compositions of the present invention may comprise hexamidine skin treatment agent at concentrations ranging from about 0.001% to about 0.1%, from about 0.005% to about 0.1%, or even from about 0.01% to about 0.1% by weight of the composition. The hexamidine skin treatment agent suitable for use herein include those aromatic diamines which generally conform to the following formula:

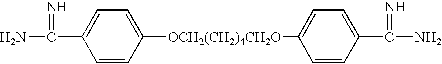

These aromatic diamines are referred to as 4,4′-[1,6-Hexanediylbis(oxy)]bisbenzenecarboximidamide; 4,4′-(hexamethylenedioxy)dibenzamidine; and 4,4′-diamidino-α,ω-diphenoxyhexane. The most popular employed form of hexamidine is the general category of hexmidine salts, which include acetate, salicylate, lactate, gluconate, tartarate, citrate, phosphate, borate, nitrate, sulfate, and hydrochloride salts of hexamidine. Specific nonlimiting examples of hexamidine salts include hexamidine isethionate, hexamidine diisethionate, hexamidine hydrochloride, hexamidine gluconate, and mixtures thereof. Hexamidine isethionate and hexamidine diisethionate are β-hydroxyethane sulfonate salts of hexamidine which are preferred for use herein as a skin treatment agent in the prevention and/or treatment of skin disorders. Hexamidine diisethionate is the most preferred hexamidine compound suitable for use as the skin treatment agent herein and is available from Laboratories Serolobilogiques (Pulnoy, France) and the Cognis Incorporation (Cincinnati, Ohio) under the tradename ELASTAB HP100.

The skin active ingredients of the present invention may comprise zinc oxide skin treatment agent at concentrations ranging from about 0.001% to about 20%, preferably from about 0.005% to about 15%, more preferably from about 0.005% to about 10%. The zinc oxide skin treatment agent can be included in the compositions as an individual zinc oxide compound or a combination of zinc oxides, provided that the individual or combined zinc oxide can readily combine with the hexamidine and niacinamide skin treatment agents to provide skin care benefits.

The zinc oxide skin treatment agent suitable for use herein include those inorganic white and yellowish-white powders that conform to the formula ZnO, and that are more fully described in The Merck Index, Eleventh Edition, entry 10050, p. 1599 (1989). Some particularly useful forms of zinc oxide include those that are manufactured and commercially available in average particle size diameters that range from about 1 nm (nanometer) to about 10 μm (micrometer), alternatively from about 10 nm to about 1 μm or even from about 20 nm to about 500 nm. Surprisingly, the inventors have discovered that the use of the above mentioned, relatively small nanoparticle diameter size zinc oxide avoids undesirable skin or hair whitening that results from the transfer of the zinc oxide containing emollient from the topsheet of absorbent article to the wearer's body during product use. This is a particular benefit when the product is a panty liner, sanitary napkin, incontinence brief, or other absorbent article intended to be used by adults having hair in the region where the lotion composition will transfer.

Commercially available zinc oxides include the white zinc oxide powders sold under the tradename ULTRAFINE 350 which is commercially available from the Kobo Incorporation located in South Plainfield, N.J. Other suitable zinc oxide materials include a premix of zinc oxide and a dispersing agent such as polyhydroxystearic acid wherein this premix is available from the Uniqema Incorporation (Wilimington, Del.) under the tradename Arlecel® P100; and a premix of zinc oxide and an isononyl isononanoate dispersing agent which is available from the Ikeda Incorporation (Island Park, N.Y.) under the tradename Salacos® 99.

The skin active ingredients of the present invention may comprise niacinamide skin treatment agent as an individual niacinamide or as a combination of niacinamides at a total niacinamide concentration ranging from about 0.01% to about 10%, preferably from about 0.05% to about 5%, more preferably from about 0.2% to about 2% by weight of the lotion composition. The niacinamide skin treatment agent provides for skin conditioning benefits as well as providing for increased efficacy of the skin treatment agents in controlling skin disorders.

Nonlimiting examples of niacinamide skin treatment agents suitable for use in the lotion compositions of the present invention include those niacinamide compounds that are amide derivatives of nicotinic acid, and that generally conform to the following formula:

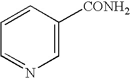

Niacinamide and nicotinic acid are also known as Vitamin B₃ and Vitamin B₅, whereas niacinamide is the commonly used active form. Niacinamide derivatives including salt derivatives are also suitable for use herein as a skin treatment agent. Nonlimiting specific examples of suitable niacinamide derivatives include nicotinuric acid and nicotinyl hydroxamic acid.

The niacinamide skin treatment agent can also be included in the composition as acidified niacinamide compounds. The process of acidifying niacinamide compounds is within the gambit of those skilled in the art, wherein one such technique involves dissolving niacinamide in an alcohol solution, adding while stirring an equal molar amount of a fatty acid such as stearic acid (e.g., mixing 1 part niacinamide to 2.4 parts stearic acid), and then air drying the mixture until the alcohol evaporates. A suitable stearic acid compound that can be used in the process of acidifying niacinamide is stearic acid sold under the tradename Emersol® 150 which is available from the Cognis Corporation.

Examples of the above niacinamide compounds are well known in the art and are commercially available from a number of sources, for example, the Sigma Chemical Company (St Louis, Mo.); ICN Biomedicals, Incorporation (Irvin, Calif.); Aldrich Chemical Company (Milwaukee, Wis.); and Em Industries HHN (Hawthorne, N.Y.).

Optional Skin Active Components:

Nonlimiting examples of optional suitable skin treatment actives useful in the present invention include allantoin; aluminum hydroxide gel; calamine; cysteine hydrochloride; racemic methionine; sodium bicarbonate; Vitamin C and derivatives thereof; protease inhibitors including serine proteases, metalloproteases, cysteine proteases, aspartyl proteases, peptidases, and phenylsulfonyl fluorides; lipases; esterases including diesterases; ureases; amylases; elastases; nucleases; guanidinobenzoic acid and its salts and derivatives; herbal extracts including chamomile; and mixtures thereof. Guanidinobenzoic acid and its salts and derivatives are more fully described in U.S. Pat. No. 5,376,655, issued to Imaki et al. on Dec. 27, 1994. These other suitable skin treatment actives are typically included at concentrations ranging from about 0.001% to about 10% by weight of the lotion composition.

Furthermore, one or more optional components known or otherwise effective for use in lotion compositions may be included provided that the optional components are physically and chemically compatible with the essential skin treatment and carrier components, or do not otherwise unduly impair product stability, aesthetics, or performance. Such optional components are typically included at concentrations ranging from about 0.001% to about 20% by weight of the compositions, and include materials such as water, skin conditioning agents, perfumes, deodorants, opacifiers, astringents, preservatives, emulsifying agents, film formers, stabilizers, proteins, lecithin, urea, colloidal oatmeal, pH control agents, and other Monographed materials that are deemed safe by the U.S. Food and Drug Administration (FDA) under 21 C.F.R. §347 for use on human skin. Other optional components for use in the lotion compositions of the present invention include fats or oils, or essential oils. These oils can be present at concentrations ranging from about 0.0001% to 10% by weight of the compositions, and include materials such as Anise Oil, Balm Mint Oil, Bee Balm Oil, Birch Oil, Bitter Almond Oil, Bitter Orange Oil, Calendula Oil, California Nutmeg Oil, Caraway Oil, Chamomile Oil, Cinnamon Oil, Cloveleaf Oil, Clove Oil, Coriander Oil, Cypress Oil, Eucalyptus Oil, Fennel Oil, Gardenia Oil, Geranium Oil, Ginger Oil, Grapefruit Oil, Hyptis Oil, Juniper Oil, Kiwi Oil, Laurel Oil, Lavender Oil, Lemongrass Oil, Lemon Oil, Lovage Oil, Mandarin Orange Oil, Musk Rose Oil, Nutmeg Oil, Olibanurn, Orange Flower Oil, Orange Oil, Peppermint Oil, Pine Oil, Rose Hips Oil, Rosemary Oil, Rose Oil, Rue Oil, Sage Oil, Sandalwood Oil, Sassafras Oil, Spearmint Oil, Sweet Marjoram Oil, Sweet Violet Oil, Tea Tree Oil, Thyme Oil, Wild Mint Oil, Yarrow Oil, Ylang Ylang Oil, Apricot Kernel Oil, Avocado Oil, Babassu Oil, Borage Seed Oil, Butter, C12-C1. Acid Triglyceride, Camellia Oil, Canola Oil, Caprylic/Capric/Lauric Triglyceride, Caprylic/Capric/Linoleic Triglyceride, Caprylic/Capric/Stearic Triglyceride, Caprylic/Capric305 Triglyceride, Carrot Oil, Cashew Nut Oil, Castor Oil, Cherry Pit Oil, Cocoa Butter, Coconut Oil, Cod Liver Oil, Corn Germ Oil, Corn Oil, Cottonseed Oil, C10-C1 Triglycerides, Evening Primrose Oil, Glyceryl Triacetyl Hydroxystearate, Glyceryl Triacetyl Ricinoleate, Glycosphingolipids, Grape Seed Oil, Hazelnut Oil, Human Placental Lipids, Hybrid Safflower Oil, Hybrid Sunflower Seed Oil, Hydrogenated Castor Oil, Hydrogenated Coconut Oil, Hydrogenated Cottonseed Oil, Hydrogenated C2-C1 Triglycerides, Hydrogenated Fish Oil, Hydrogenated Lard, Hydrogenated Menhaden Oil, Hydrogenated Mink Oil, Hydrogenated Orange Roughy Oil, Hydrogenated Palm Kernel Oil, Hydrogenated Palm Oil, Hydrogenated Peanut Oil, Hydrogenated Shark Liver Oil, Hydrogenated Soybean Oil, Hydrogenated Tallow, 315 Hydrogenated Vegetable Oil, Lard, Lauric/Palmitic/Oleic Triglyceride, Lanolin and Lanolin derivatives, Lesquerella Oil, Macadamia Nut Oil, Maleated Soybean Oil, Marula Oil, Meadowfoarn Seed Oil, Menhaden Oil, Mink Oil, Moringa Oil, Mortierella Oil, Oleic/Linoleic Triglyceride, Oleic/Paimitic/Lauric/Myristic/Linoleic Triglyceride, Oleostearine, Olive Husk Oil, Olive Oil, Ornental Lipids, Palm Kernel Oil, Palm Oil, 320 Peach Kernel Oil, Peanut Oil, Pentadesma Butter, Phospholipids, Pistachio Nut Oil, Rapeseed Oil, Rice Bran Oil, Safflower Oil, Sesame Oil, Shark Liver Oil, Shea Butter, Soybean Oil, Sphingolipids, Sunflower Seed Oil, Sweet Almond Oil, Tall Oil, Tallow, Tribehenin, Tricaprin, Tricaprylin, Triheptanoin, C10 Fatty Acids: Arachidic Acid, Behenic Acid, Capric Acid, Caproic Acid, 330 Caprylic Acid, Coconut Acid, Corn Acid, Cottonseed Acid, Hydrogenated Coconut Acid, Hydrogenated Menhaden Acid, Hydrogenated Tallow Acid, Hydroxystearic Acid, Isostearic Acid, Lauric Acid, Linoleic Acid, Linolenic Acid, Myristic Acid, Oleic Acid, Palmitic Acid, Palm Kernel Acid, Pelargonic Acid, Ricinoleic Acid, Soy Acid, Stearic Acid, Tallow Acid, Undecanoic Acid, Undecylenic Acid, Wheat Germ Acid, and the like, as well as mixtures thereof. Specific optional lotion conditioning agents found useful in the present invention include panthenol, glycerine, and chamomile oil which are described in detail hereinbelow.

Panthenol:

Where included, panthenol typically comprises from about 0.001% to about 10%, preferably from about 0.005% to about 5%, more preferably from about 0.05% to about 2% by weight of the lotion composition. The optional panthenol skin conditioning agent provides for skin emolliency benefits that can leave the skin feeling smooth, soothing, and soft during and after interaction of the skin tissues with the skin treatment agents. The lotion compositions of the present invention can include an individual panthenol compound or a mixture of panthenol compounds.

Nonlimiting examples of panthenol include those panthenol compounds which are alcohol or ester derivatives of pantothenic acid. Pantothenic acid is a member of the B complex family and is often referred to as Vitamin B₃. Like pantothenic acid, the panthenol alcohol derivatives of this acid can exist as stereoisomers, for example, the D(+) form, the L(−) form, the racemate, and mixtures of the D(+) and L(−) forms. Specific examples of panthenol include, but are not limited to, D-panthenol (a.k.a. dexpanthenol), and dl-panthenol. Panthenol is more fully described in The Merck Index, Eleventh Edition, entry 2924, p. 464 (1989), which description is incorporated herein by reference. Examples of commercially available panthenol include D-panthenol which is available from Roche Vitamins Incorporation (Nutley, N.J.), a subsidiary of F. Hoffman LaRoche, Ltd.

Glycerine:

Where included, the lotion compositions comprise the preferred optional glycerine skin conditioning agent at concentrations ranging from about 0.01% to about 10%, preferably from about 0.02% to about 5%, more preferably from about 0.05% to about 2% by weight of the lotion composition. The optional glycerine skin conditioning agent also provides for skin emolliency benefits such as smooth, soothing, and soft feeling skin, as well as being a dispersing agent for the niacinamide skin treatment agent.

Glycerine is a C3 monohydric alcohol that is also referred to as glycerol and 1,2,3-propanetriol. Glycerine derivatives are also suitable for use as an optional skin conditioning agent herein wherein such derivatives include polyglycerols having from about 2 to about 16 repeating glycerol moieties. A specific example of a suitable glycerine skin conditioning agent is Glycerine, USP Kosher® which is commercially available from the Procter & Gamble Company located in Cincinnati, Ohio.

Chamomile:

The lotion compositions comprise the preferred optional chamomile oil at concentrations ranging from about 0.0001% to about 10%, preferably from about 0.001% to about 5%, more preferably from about 0.005% to about 2% by weight of the lotion composition. The optional chamomile oil skin conditioning agent also provides for skin benefits such as soothing. Chamomile oil is commonly prepared as an oil extract of chamomile flowers. An example of a commercially available chamomile oil include Phytoconcentrol Chamomile which is available from Dragoco Incorporation (Totowa, N.J.).

C. Treating Body Contacting Surface With Skin Care Composition

In preparing products treated with skin care compositions, the skin care composition is applied onto at least a portion of the body facing surface of absorbent articles by any of a variety of application methods that evenly distribute viscous materials can be used including, but not limited to printing, spraying, coating, brushing, extrusion, or combinations of these application techniques. If the skin care composition is relatively hydrophobic, then the skin care composition may not be distributed in the fluid path (such as apertures of the topsheet) to ensure the ability of the topsheet to transmit fluid to the underlying absorbent core. The skin care composition may be applied in a pattern of coating including stripes, dots, circles and the like.

The effective amount of composition disposed on the body contacting surface will depend, to a large extent, on the particular skin care composition used, a portion of the body contacting surface where the skin care composition is applied, and/or the type of absorbent article treated. Typically, a safe and effective amount of the lotion compositions of the present invention is applied to an absorbent article such that at least about 0.00015 mg/cm² (0.001 mg/in²) to about 15.5 mg/cm² (100 mg/in²), preferably from about 0.0006 mg/cm² (0.004 mg/in²) to about 11 mg/cm² (72 mg/in²), more preferably from about 0.005 mg/cm² (0.03 mg/in²) to about 6.2 mg/cm² (40 mg/in²), of the composition is transferred to the skin during a single use of an absorbent article which is typically about a three hour period. These ranges are by way of illustration only and the skilled artisan will recognize that the nature of the composition will dictate the level that must be disposed thereon to achieve the desired skin effects, and that such levels are ascertainable by routine experimentation in light of the present disclosure.

Absorbent articles are generally changed every three to six hours during the day and once for overnight protection, resulting in at least a safe and effective amount of from about 0.00045 mg/cm² (0.003 mg/in²) to about 124 mg/cm² (800 mg/in²), preferably from about 0.0018 mg/cm² (0.012 mg/in²) to about 88 mg/cm² (576 mg/in²), more preferably from about 0.015 mg/cm² (0.09 mg/in²) to about 49.6 mg/cm² (320 mg/in²), of the lotion composition being administered within a one day interval (24 hour period). However, the transfer of the lotion compositions of the present invention onto a wearer's skin via an absorbent article described herein can occur for one day, several days, weeks, months, or years at appropriate intervals provided that safe and effective amounts of the lotion compositions are administered to deliver the skin treatment benefits described herein.

Any suitable method can be used in determining the amount of a lotion composition described herein that is transferred to the skin of a wearer during use of an absorbent article containing the composition. An example of specific methods for the calculation of transfer amounts of lotion compositions include Gas Chromatographic and other quantitative analytical procedures that involve the analysis of in vivo skin analog materials. A suitable Gas Chromatographic procedure is more fully described in WO 99/45973, Donald C. Roe et al, published Sep. 16, 1999.

The skin care composition is typically applied from a melt thereof to the article. Since the skin care composition melts at significantly above ambient temperatures, it is usually applied as a heated coating to the body contacting surface of the article. The temperature is determined considering primarily the melting point of the skin care composition and the other factors such as lowering of the temperature in the manufacturing process of the article. The skin care composition is often heated to a temperature in the range from 50° C. to 100° C., more often from 60° C. to 90° C., prior to being applied to the article. Once the melted skin care composition has been applied to the article, it is allowed to cool and solidify to form solidified coating or film on the surface of the body contacting surface. The application process is designed to aid in the cooling/set up of the skin care composition Examples of applying the skin care composition to the body contacting surface is described in, e.g., U.S. Pat. No. 5,643,588, “Diaper Having a Lotioned Topsheet”, issued to Roe, Bakes & Warner on Jul. 1, 1997; Representative topsheets treated with a skin care composition are described in, e.g., U.S. Pat. No. 5,643,588, “Diaper Having a Lotioned Topsheet”, issued to Roe, Bakes & Warner on Jul. 1, 1997; and U.S. Pat. No. 5,635,191, “Diaper Having a Lotioned Topsheet Containing a Polysiloxane Emollient”, issued to Roe & Mackey on Jun. 3, 1997; U.S. Pat. No. 5,609,587, “Diaper Having a Lotioned Topsheet Comprising a Liquid Polyol Polyester Emollient and an Immobilizing Agent”, issued to Roe on Mar. 11, 1997; and U.S. Pat. No. 5,607,760, “Disposable Absorbent Article Having a Lotioned Topsheet Containing an Emollient and a Polyol Polyester Immobilizing Agent”, issued to Roe, on Mar. 4, 1997. Methods for delivering a skin care composition via the repeated use of absorbent articles having such treated topsheets are disclosed in U.S. Pat. No. 6,803,496 entitled “A Method For Maintaining or Improving Skin Health”, Elder, et al., issued on Oct. 12, 2004; U.S. Pat. No. 6,710,223 entitled “A Method For Improving Skin Condition”, Van Rijswijck, et al. issued on Mar. 24, 2004; and U.S. patent application Ser. No. 08/908,852 “Diaper Having A Lotioned Topsheet”, Roe, et al. filed on Aug. 8, 1997. Representative cuffs treated with a skin care composition are described in, e.g., U.S. Pat. No. 6,156,024application Ser. No. 08/766,386 “Absorbent Articles Having Lotioned Leg Cuffs”, Schulte et al, issued on Dec. 5, 2000; PCT publication WO 98/47546 entitled “Absorbent Articles Having Lotioned Leg Cuffs Containing a Polysiloxane Emollient”, published by Schulte et al, on Oct. 29, 1998; U.S. Pat. No. 6,166,285 entitled “Absorbent Article Having cuffs and Topsheet with Skin Care Composition Disposed Thereon”, issued to Schulte et al, on Dec. 26, 2000; U.S. Pat. No. 6,120,488 entitled “Absorbent Article Having cuffs and Topsheet with Skin Care Composition(s) Disposed Thereon”, issued to VanRijswijck, et al, on Sep. 19, 2000. Representative interlabial absorbent articles treated with a skin care composition are disclosed in, e.g., U.S. Pat. No. 6,409,713 “Emollient-Treated Absorbent Interlabial Device”, issued to Osborn et al, on Jun. 25, 2002; U.S. Pat. No. 5,891,126 “Absorbent Interlabial Device Treated With A Polysiloxane Emollient”, issued to Osborn et al, on Apr. 6, 1999. Representative absorbent articles having breathability treated with a skin care composition are disclosed in, e.g., U.S. patent application Ser. No. 6,107,537 entitled “Disposable Absorbent Articles Providing a Skin Condition Benefit”, issued to Elder, et al., Aug. 22, 2000.

D. Barrier Sheet

The barrier sheet is resistant to visually perceptible transfer of the skin care composition onto itself. The barrier sheet is resistant to transfer of the skin care composition, such that if the skin care composition comes into contact with the barrier sheet, such as when the article is folded upon itself, a visually perceptible stain or spot from the oil-based skin care composition does appear on the barrier sheet. The barrier sheet can be resistant to either tactile or visually perceptible transfer of the skin care composition thereon. The barrier sheet can also be treated to reduce the migration of the skin care composition there through.

The barrier sheet can comprise a base sheet. The base sheet may be any material, such as a paper or a film. The base sheet may be treated with a composition comprising a component selected from the group consisting of hydrophilic polymers, silicone, and mixtures thereof. The base sheet can also be treated with a composition comprising a component selected from the group consisting of fluorochemicals, hydrophilic polymers, inorganic particles, or mixtures thereof. In one embodiment base sheet is fluorochemical free. The base sheet may be prepared from pulp to render it oil-resistant without any additional chemical treatment.

The barrier sheet may be treated to be releasable, when the barrier sheet is used to cover adhesives. For this purpose, the barrier sheet may be treated with a composition that comprises a component selected from the group consisting of silicone, a wide variety of fluorochemicals, or mixtures thereof. In one embodiment barrier sheet is fluorochemical free. Examples of barrier sheets exhibiting resistant to transfer of a skin care lotion include Greaseproof Release Paper GR/3786-51 from Papertec, Inc (Elizabeth, N.J.), or Nordic Paper Eco Bake #BP 118 from Central National Gottesman (Purchase, N.Y.), or Nordic Paper Silidor 1 #BP114 from Central National Gottesman (Purchase, N.Y.), or Greaseproof Paper #238-2307 from Channeled Resources Group (Chicago, Ill.). The barrier can a basis weight of between 10 g/m² and 200 g/m², preferably 15 g/m² between 100 g/m², and more preferably between 20 g/m² and 50 g/m².

Fluorochemicals, if used, may be selected from the group consisting of polytetrafluoroethylene, polyfluorinated ethylene propylene, perfluoroalkyl acrylate, polyperfluoroalkoxy, polyhexafluoropropylene, polyhexafluoroisobutylene, polychlorotrifluoroethylene, polyvinylidene fluoride, polyvinyl fluoride, fluoroalkyl salt, copolymers of ethylene, copolymers of propylene, or mixtures thereof. Fluorochemical may be applied to the base sheet of the barrier sheet; in the form of solution or dispersion in water, organic solvent(s) such as alcohol, or mixture of them; or in the form of molten resin. If fluorochemical is applied to the base sheet in the form of solution or dispersion, it may be applied by, e.g., (a) immersing the base sheet in the solution or dispersion then drying, (b) spraying the solution or dispersion onto the base sheet then drying, or (c) coating the base sheet by printing or painting with a brush or a roller then drying. If fluorochemical is applied to the base sheet in the form of molten resin, it may be applied by extrusion laminating. Fluorochemicals that can be used in the barrier sheet include fluoroalkyl salt, for example, fluoroalkyl ammonium salt dissolved in water. Fluoroalkyl ammonium salt solved in water is available under the trade name of Scotchban FC-807A by 3M, Haven 1005, Canadastraat 11, B-2070, Zwijndrecht, Belgium.

Hydrophilic polymers may be selected from the group consisting of polyvinyl alcohol, polyethylene vinyl alcohol, polyvinyl acetate, polyethylene vinyl acetate, polyacrylate, polyethylene acrylate, polymethacrylate, polyethylene methacrylate, polyesters, polyethers, polyimide, polyamide, or mixtures thereof. Hydrophilic polymer may be applied to the base sheet of the barrier sheet in the form of solution or dispersion in water, organic solvent(s) such as alcohol, or mixture of them. If hydrophilic polymer is applied to the base sheet in the form of solution or dispersion, it may be applied by, e.g., (a) immersing the base sheet in the solution or dispersion then drying, (b) spraying the solution or dispersion onto the base sheet then drying, or (c) coating the base sheet by printing or painting with a brush or a roller then drying. If hydrophilic polymer is applied to the base sheet in the form of molten resin, it may be applied by extrusion laminating. One hydrophilic polymer that can be used in the barrier sheet is polyethylene vinyl acetate and is available under the trade name of ELVAX by DuPont, 1007 Market Street Wilmington, Del. 19898.

Inorganic particles may be selected from the group consisting of kaolin clay, bentonite clay, montmorillonite clay, hectorite clay, tarc, silica, fumed silica, or mixtures thereof. They may be applied to the base paper with some binders. The binder may be selected from the group consisting of polyvinyl alcohol, polyethylene vinyl alcohol, polyvinyl acetate, polyethylene vinyl acetate, polyacrylate, polyethylene acrylate, starch, tragacanth gum, guar gum, arabic gum, karaya gum, dextrin, natural resin, thermoplastic rubber (such as isoprene rubber, butadiene rubber, neoprene rubber, styrene rubber, styrene isoprene rubber, styrene butadiene rubber) based adhesives, or mixtures thereof. If the binder is a thermoplastic rubber based adhesive, the inorganic particles may be applied to the base sheet by, e.g. super calendaring. If the binder is water soluble, the inorganic particles may be applied to the base sheet by, e.g., printing or painting slurry composed of the inorganic particle, the binder, and small amount (up to 20%) of water onto the base sheet with a brush or a roller. The inorganic particles, such as kaolin clay, bentonite clay, montmorillonite clay, hectorite clay, or mixtures thereof, may be combined with thermoplastic rubber based adhesive as the binder.

### EXAMPLE 1 OF THE PRESENT INVENTION

A barrier sheet can be a Greaseproof Paper GR/3786-51 from Papertec, Inc (Elizabeth, N.J.) paper having basis weight of 57 g/m². The entirety of one surface of the paper is also treated with silicone such that the surface becomes releasable with adhesive.

A sanitary napkin with wings and a skin care composition applied to the topsheet thereof can be tri-folded and wrapped in a package comprising a flexible film wrapper sheet made of low density polyethylene film having a thickness of 40 μm. The barrier sheet can serve as the adhesive cover for the panty fastening adhesive on the wings, as well as a central pad adhesive cover for the panty fastening adhesive on the main body of the pad. The flap of the treated article is folded onto the topsheet. The main wrapper sheet, the flap adhesive cover, the central pad adhesive cover are assembled as shown in FIGS. 5-9.

### EXAMPLE 2 OF THE PRESENT INVENTION

A barrier sheet can be a greaseproof paper of Nordic Paper Eco Bake #BP118 from Central National Gottesman (Purchase, N.Y.) having basis weight of 39 g/m². A portion of one surface of the barrier sheet is treated with silicone such that the surface becomes releasable with adhesive.

A sanitary napkin with wings and a skin care composition applied to the topsheet thereof can be tri-folded and wrapped in a package comprising a flexible film wrapper sheet made of low density polyethylene film having a thickness of 40 μm. The barrier sheet can serve as the adhesive cover for the panty fastening adhesive on the wings, as well as a central pad adhesive cover for the panty fastening adhesive on the main body of the pad. The flap of the treated article is folded onto the topsheet. The main wrapper sheet, the flap adhesive cover, the central pad adhesive cover are assembled as shown in FIGS. 5-10.

### EXAMPLE 3 OF THE PRESENT INVENTION

A barrier sheet can be a greaseproof paper of Nordic Paper Silidor 1 #BP114 from Central National Gottesman (Purchase, N.Y.) having basis weight of 41 g/m². One surface of the paper is also treated with silicone such that the surface becomes releasable with adhesive.

A sanitary napkin with wings and a skin care composition applied to the topsheet thereof can be tri-folded and wrapped in a package comprising a flexible film wrapper sheet made of low density polyethylene film having a thickness of 40 μm. The barrier sheet can serve as the adhesive cover for the panty fastening adhesive on the wings, as well as a central pad adhesive cover for the panty fastening adhesive on the main body of the pad. The flap of the treated article is folded onto the topsheet. The main wrapper sheet, the flap adhesive cover, the central pad adhesive cover are assembled as shown in FIGS. 5-10.

All documents cited in the Detailed Description of the Invention are, are, in relevant part, incorporated herein by reference; the citation of any document is not to be construed as an admission that it is prior art with respect to the present invention.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. An absorbent article comprising a skin care composition and a barrier sheet, wherein
the barrier sheet is fluorochemical free and resistant to the visually-perceptible transfer of said skin care composition thereon.

2. The absorbent article of claim 1 wherein the barrier sheet is disposed to cover a portion of the absorbent article.

3. The absorbent article of claim 2 wherein the absorbent article has an adhesive, wherein the barrier sheet is disposed to cover the adhesive.

4. The absorbent article of claim 3 wherein the adhesive is provided on the garment surface of the absorbent article, wherein the barrier sheet covers the adhesive before the use of the absorbent article and is removed when in use.

5. The absorbent article of claim 4 wherein the absorbent article has flaps extending outwardly, and the adhesive is a flap adhesive provided on at least a portion of the garment surface of the flap to secure the flap to the wearer's undergarment when the absorbent article is used.

6. The absorbent article of claim 5 wherein the skin care composition is provided on at least a portion of the body surface of the absorbent article, the flap is folded over the body surface of the absorbent article before use of the absorbent article, wherein the barrier sheet is disposed to cover the flap adhesive.

7. The absorbent article of claim 6 wherein the absorbent article is folded before use of the absorbent article such that the barrier sheet faces at least a portion of the body surface which is provided with a skin care composition.

8. The absorbent article of claim 4 wherein the adhesive is a pad adhesive provided on at least a portion of the garment surface of the absorbent article to secure the absorbent article to the wearer's undergarment when the absorbent article is used.

9. The absorbent article of claim 8 wherein the skin care composition is provided on at least a portion of the body surface of the absorbent article, wherein the barrier sheet is disposed to cover the pad adhesive.

10. The absorbent article of claim 9 wherein the absorbent article is folded before use of the absorbent article such that the barrier sheet faces at least a portion of the body surface which is provided with a skin care composition.

11. The absorbent article of claim 1 wherein the absorbent article has a main wrapper sheet to wrap the absorbent article individually, wherein at least a portion of the wrapper comprises the barrier sheet.

12. The absorbent article of claim 1 wherein the barrier sheet comprises a base sheet, wherein the base sheet is treated with a composition comprising a component selected from the group consisting of hydrophilic polymers, silicone and mixtures thereof.

13. The absorbent article of claim 12 wherein the barrier sheet is comprised of two surfaces; one surface being treated to be releasable.

14. The absorbent article of claim 12 wherein the hydrophilic polymer is a component selected from the group consisting of polyvinyl alcohol, polyethylene vinyl alcohol, polyvinyl acetate, polyethylene vinyl acetate, polyacrylate, polyethylene acrylate, polymethacrylate, polyethylene methacrylate, polyesters, polyethers, polyimide, polyamide, or mixtures thereof.
